(19) United States
(12) Patent Application Publication
(10) Pub. No.: US 2010/0256704 A1
(43) Pub. Date: Oct. 7, 2010

(54) Utilizing electrical stimulation in response to acceleration

(75) Inventors: Lance L. Annicelli, Bolling AFB, DC (US); James Kisner, San Antonio, TX (US); ULF Balldin, San Antonio, TX (US)

Correspondence Address: DEPARTMENT OF THE AIR FORCE, AFMC LO/JAZ, BLdg 11, Room D18, WRIGHT-PATTERSON AFB, OH, 45433-7109, US

(73) Assignee: United States of America, as represented by the Secretary of the Air Force

(21) Appl. No.: 11/417,288

(22) Filed: Apr. 14, 2006

(51) Int. Cl. A61N 1/36

(52) U.S. Cl. 607/48

(57) ABSTRACT

One example of a solution provided here comprises sensing acceleration, and electrically stimulating one or more muscle groups of a person subject to the acceleration. The stimulating is accomplished automatically. Other examples include systems, kits and garments.

### RIGHTS OF THE GOVERNMENT

The invention described herein may be manufactured and used by or for the Government of the United States for all governmental purposes without the payment of any royalty.

### BACKGROUND OF THE INVENTION

The invention relates generally to aerospace technology, and more specifically to solutions for problems encountered by humans subjected to acceleration.

During high acceleration, with a crew member's head aimed toward the inside of a turn, hydrostatic forces on the person's blood column may cause pooling of blood in peripheral body regions and reduction of the cerebral blood pressure to critically low levels. This creates loss of peripheral and central vision and ultimately a G-induced loss of consciousness (G-LOC) with a possibly fatal outcome. (“G” is shorthand for “acceleration.”) The conventional approach is to use the Anti-G Straining Maneuvers (AGSM) along with the conventional anti-G suit. AGSM involves lower muscle tensing and cyclic breathing. The conventional anti-G suit has pneumatic bladders over the abdomen and legs that exert pressure on adjacent tissues to minimize pooling of blood.

The conventional approach might not have a response time short enough to cope with the rapid onset of G forces. The conventional anti-G suit has considerable bulk and weight, imposing a thermal burden on the aircrew. The conventional anti-G suit depends on the aircraft's oxygen system. AGSM depends on physical training and voluntary effort by the aircrew, and is a distraction. Performing the AGSM during high acceleration is extremely fatiguing. Relying on the conventional approach may lead to pilot fatigue, exhaustion, G-LOC, possibly mission failure, loss of aircraft, or even loss of life.

Thus there is a need for solutions for responding to G forces, while alleviating or avoiding the above-mentioned problems.

### SUMMARY OF THE INVENTION

One example of a solution provided here comprises sensing acceleration, and electrically stimulating one or more muscle groups of a person subject to the acceleration. The stimulating is accomplished automatically. Other examples include systems, kits and garments.

### BRIEF DESCRIPTION OF THE DRAWINGS

FIG. 1: this simplified drawing illustrates an example of a system according to the present invention.

FIG. 2: this flow chart illustrates an example of a method according to the present invention.

FIG. 3: this drawing illustrates an example of a garment according to the present invention.

FIG. 4: this drawing illustrates an example of means for stimulating abdominal muscles.

FIG. 5: this drawing illustrates an example of means for stimulating some lower extremity muscle groups.

FIG. 6: this drawing shows an example of a garment (outside front view).

FIG. 7: this drawing shows an example of a garment (inside front view).

FIG. 8: this drawing shows an example of a garment (inside rear view).

### DETAILED DESCRIPTION

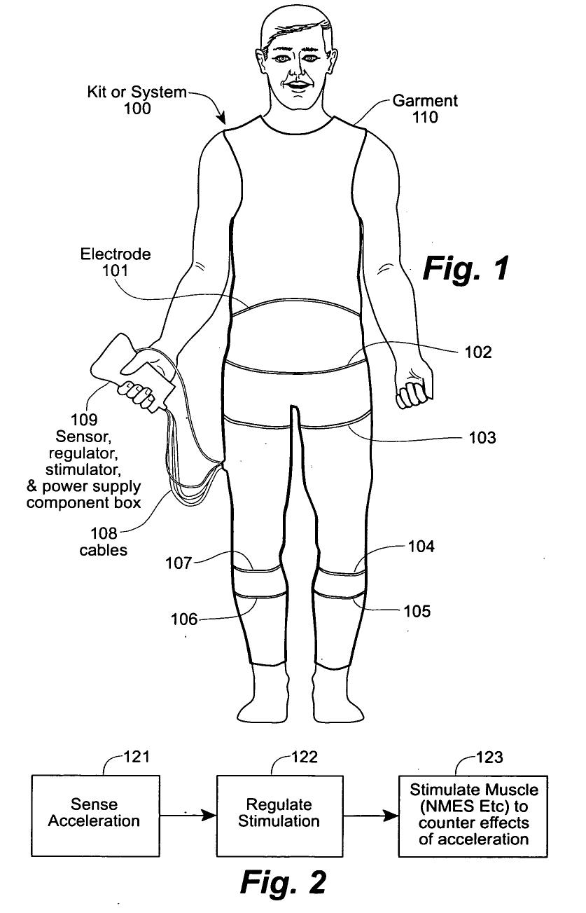

FIGS. 1-2 show examples of a system and method according to the present invention. Beginning with the method aspect, FIG. 2 summarizes an example of a method of responding to acceleration, in a short flow chart (Blocks 121-123), which may be considered with features of FIG. 1. The method aspect comprises sensing acceleration (Block 121) and electrically stimulating (Block 123) one or more muscle groups of a person subject to the acceleration, thereby countering adverse physical effects of the acceleration. The stimulation is accomplished automatically. Electrically stimulating muscles is expected to produce an increase in blood pressure, a mechanical decrease in peripheral blood pooling, and an increased tolerance for acceleration.

Taking a closer look at this example, Block 121 represents sensing acceleration, employing an acceleration sensor in component box 109. Block 122 represents automatically regulating stimulation, in direct relation to the magnitude of acceleration (employing a regulator in box 109). Block 123 represents electrically stimulating one or more muscle groups (typically including lower extremity muscle groups). These muscle groups are selected from abdominal muscles (electrode location 101), gluteal muscles (not shown), thigh muscles (electrode location 103), and calf muscles (electrode locations 105 and 106).

Continuing with details represented by Block 123, in response to sensing acceleration, an effective amount of electrical stimulation is administered for countering adverse physical effects of acceleration. The examples given here include administering an effective amount of electrical stimulation to raise the blood pressure of the person.

Neuromuscular electrical stimulation (NMES) is commonly used by physical therapists. The examples in this patent application involve NMES. However, some other mode or protocol for electrical stimulation could be used. The method in Blocks 121-123 may be utilized as automatic contraction enhancement, together with an anti-G straining maneuver. The method in Blocks 121-123 may be utilized together with pneumatic compression. Background information on acceleration effects on humans, and conventional approaches to countering these effects, are provided by Dr. Balldin's “Acceleration Effects On Fighter Pilots,” pages 1025-1038, in Pandoff and Burr, eds., Textbooks of Military Medicine: Medical Aspects of Harsh Environments, Vol. 2, Defense Dept., 2002 (hereby incorporated by reference).

The method summarized in Blocks 121-123 may include supplying power for stimulation, independently from any aircraft system (employing a self-contained power supply in box 109). Background information on small, battery-operated stimulators is found in: Lyons, et al., “Differences in quadriceps femoris muscle torque when using a clinical electrical stimulator versus a portable electrical stimulator.” Phys Ther. 2005; 85:44-51 (hereby incorporated by reference).

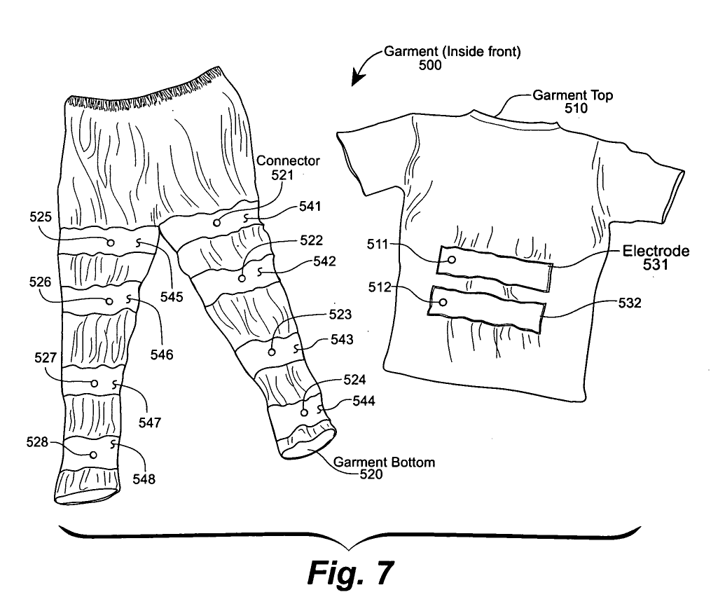

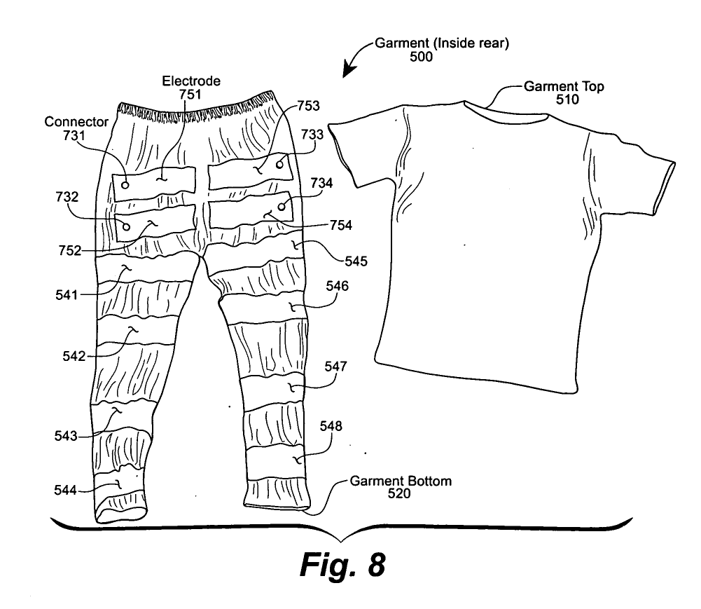

A system aspect is shown in FIG. 1. (FIGS. 1, 3, 4, and 5 show examples from an early stage of development.) FIG. 1 is a simplified diagram. The system 100 comprises means for stimulating one or more muscle groups: system 100 has electrodes numbered 101-107 for stimulating skeletal muscles. Concerning electrode placement, preferably there may be two electrodes over the calf, two over the thigh, two over the abdominal region, etc. (see detailed examples in FIGS. 7-8). Cables 108 supply electrical current, and connect electrodes 101-107 with component box 109. System 100 has a power supply, regulator, and other components in box 109. Electrodes 101-107 may be attached to, or woven into a garment 110. System 100 has means for sensing acceleration (e.g. an accelerometer) in box 109. Box 109 is shown in a user's hand, to symbolize portability. During flight, Box 109 could be attached to some point on a flight suit (not shown) or on a cockpit surface, for example.

A kit aspect also is apparent in FIG. 1. FIG. 1 illustrates an example of an acceleration-countermeasure kit, having components of system 100 that are convenient to package, sell, and use together. A kit has packaged together an acceleration sensor (in box 109), and at least one electrode (shown at 101-107) adapted for stimulating skeletal muscles.

System 100 or a kit may have a power supply (such as a battery, in box 109), independent from any aircraft system. System 100 or a kit may have a regulator (in box 109), for automatically regulating stimulation, in direct relation to the magnitude of acceleration.

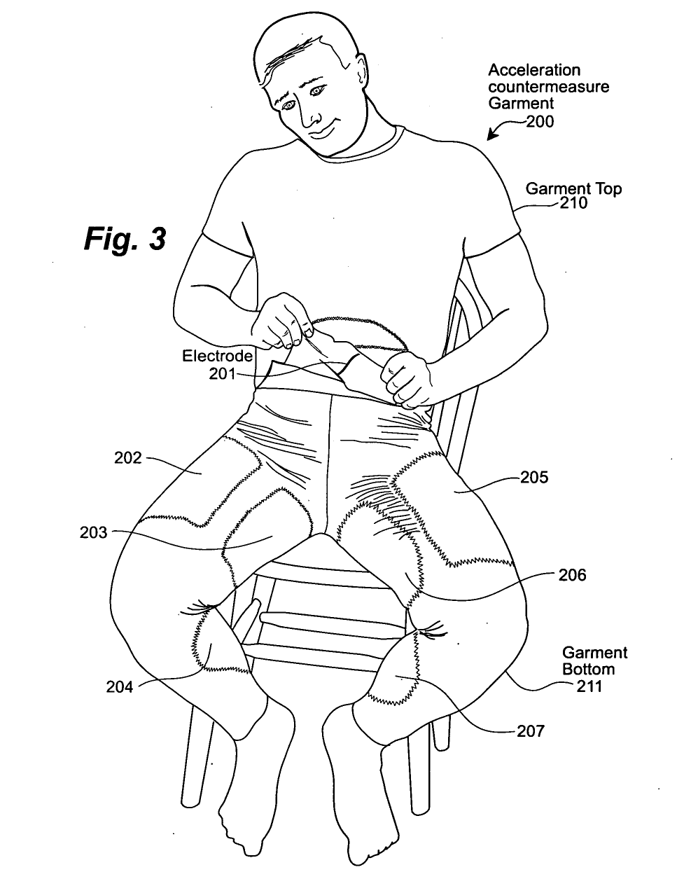

FIG. 3 shows an example of a garment 200. Garment 200 has at least one electrode (electrode locations shown at 201-207) adapted to stimulating one or more muscle groups selected from abdominal muscles, gluteal muscles, thigh muscles, and calf muscles. (Concerning electrode placement, see also detailed examples in FIGS. 7-8). For example, electrode 201 is adapted to stimulating abdominal muscles. Electrode locations 202, 203, 205, and 206 are adapted to stimulating thigh muscles. Electrode locations 203 and 206 also may be adapted to stimulating gluteal muscles. Electrode locations 204 and 207 are adapted to stimulating calf muscles. Electrodes shown at 201-207 are attached to, or woven into garment 200. Garment 200 has close-fitting fabric adapted to positioning the electrode in contact with a person's skin or adapted to wearing under a second garment (e.g. a conventional anti-G suit or flight suit). The fabric covers at least the wearer's legs. In this example, garment 200 has two pieces: top 210 and bottom 211. The inside of top 210 is exposed, showing electrode 201 attached to garment top 210. For example electrode 201 may be a kind of electrode sold under the trademark ELECTRO-MESH by Prizm Medical Inc of Duluth, Ga. Another example of a potentially useful electrode material is an electrically conductive, elastic material sold under the trademark METAL RUBBER by NanoSonic, Inc. of Blacksburg, Va.

A garment like garment 110 in FIG. 1 or garment 200 in FIG. 3 may be useful in high-performance aircraft or in spacecraft. Such a garment also may be useful in heavy or transport aircraft. Such a garment may counter adverse physical effects of long-duration flights. The muscle stimulation may help keep blood from pooling in peripheral body regions, possibly preventing Deep Vein Thrombosis (DVT), for example. For application in long-duration flights, electrical stimulation may be delivered in response to a variable other than acceleration, such as time aloft for example. An acceleration sensor, timer, and power supply in box 109, seen in FIG. 1, may serve as means for supplying electrical current to garment 110's electrodes, in response to acceleration, or trip duration, or both.

Here is a summary of potential advantages of methods, systems, and garments like those seen in FIG. 1 and FIG. 3. Potential design advantages include: short response time for instantaneous G-protection, self-contained G protection (not dependent on the aircraft's electrical or oxygen system—no G-valve or hoses), less weight and bulk, low installation expense, and utilizing one system for all aircraft. Potential physiological advantages include: decreased fatigue, improved recovery, reduced heat stress (reduced thermal burden by eliminating both bulk and weight of conventional anti-G systems), decreased venous blood pooling, ability to recruit increased muscle fibers for increased muscular contraction, and sustained muscle contraction. Potential tactical advantages include: improved situational awareness allowing the crew to concentrate on flying and deploying aircraft weapon systems, enhanced performance and increased sortie rate (associated with reduced thermal burden and decreased fatigue), enhanced flight safety, and reduced operating cost (reducing the need for onboard support systems could decrease both cost and weight of the aircraft).

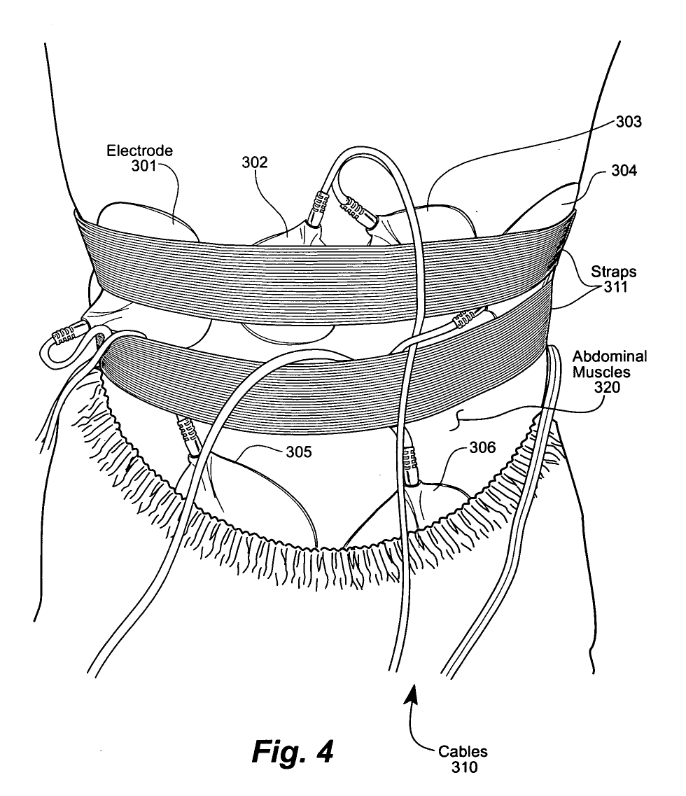

FIG. 4 shows an example of means for stimulating abdominal muscles (shown at 320). This example includes electrodes 301-306 and straps 311 for positioning electrodes. Cables 310 supply electrical current.

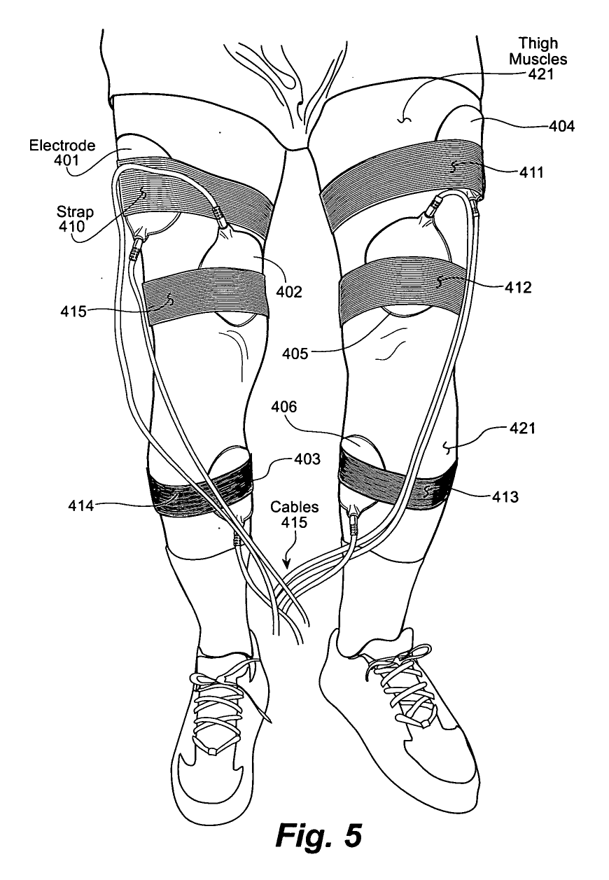

FIG. 5 shows an example of means for stimulating some lower extremity muscle groups (thigh muscles shown at 420 and calf muscles shown at 421). This example includes electrodes 401-406 and straps 410-415 for positioning electrodes. Cables 409 supply electrical current.

FIGS. 4-5 serve to illustrate a proof-of-concept example. Using electrodes like those shown in FIGS. 4-5, we accomplished the preliminary proof of concept, showing an increase in arterial blood pressure by electrically stimulating muscle groups at 1+Gz (normal gravity). An increase in blood pressure is expected to produce a decrease in peripheral blood pooling, and an increased tolerance for acceleration. For these tests the person was relaxed (no voluntary lower muscle tensing nor cyclic breathing). After several one-hour-long sessions, there were no apparent signs of exhaustion or muscle fatigue. We have found certain stimulating frequency settings produce the greatest increase in blood pressure in preliminary measurements. The most pronounced blood pressure increases recorded were 60 and 25-30 mm Hg of the systolic and diastolic pressure, respectively, with 70 and 75 Hz stimulation (See Table 1 below).

TABLE 1
Electrical muscle stimulation: average arterial blood pressure
increase from baseline in preliminary recordings. Mean
systolic/diastolic (mmHg)

| | | |
|---|---|---|
| Russian stim 2500 Hz (16 recordings in 4 subjects) | 5 | 1 |
| Stim 200 Hz (16 recordings in 3 subjects) | 6 | 3 |
| Stim 100 Hz (15 recordings in 5 subjects) | 19 | 8 |
| Stim 75 Hz (7 recordings in 2 subjects) | 29 | 14 |
| Stim 70 Hz (13 recordings in 5 subjects) | 28 | 12 |
| Stim 60 Hz (4 recordings in 2 subjects) | 9 | 8 |
| Stim 50 Hz (10 recordings in 3 subjects) | 28 | 11 |
| Tens 39 Hz (13 recordings in 5 subjects) | 13 | 7 |
| Tens 30 Hz (3 recordings in 1 subject) | 13 | 6 |
| Tens 10 Hz (1 recording in 1 subject) | −8 | −5 |
| Muscle strain only (3 recordings in 3 subjects) | 26 | 22 |
| Muscle strain and stim (2 recordings in 1 subject) | 28 | 24 |

Background information on various frequencies and regimens for electrical stimulation, including Russian electrical stimulation (Russian stim) is provided by Ward and Shkuratova in “Russian electrical stimulation: the early experiments,” Phys Ther. 2002; 82:1019-1030 (hereby incorporated by reference). TENS is an acronym for “transcutaneous electrical nerve stimulation”.

For these proof-of-concept tests we recorded blood pressure with the use of a continuous blood pressure measuring device (sold under the trademark PORTAPRES by Finapres Medical Systems BV). We used an electrical stimulator (sold under the trademark TAMTEC PRO TDR68, by TONE-A-MATIC International Incorporated, serial number 0100296) along with electrodes and straps like those shown in FIGS. 4-5. We also used other electrodes sold under the trademark ELECTRO-MESH by Prizm Medical Inc of Duluth, Ga. (see FIG. 2).

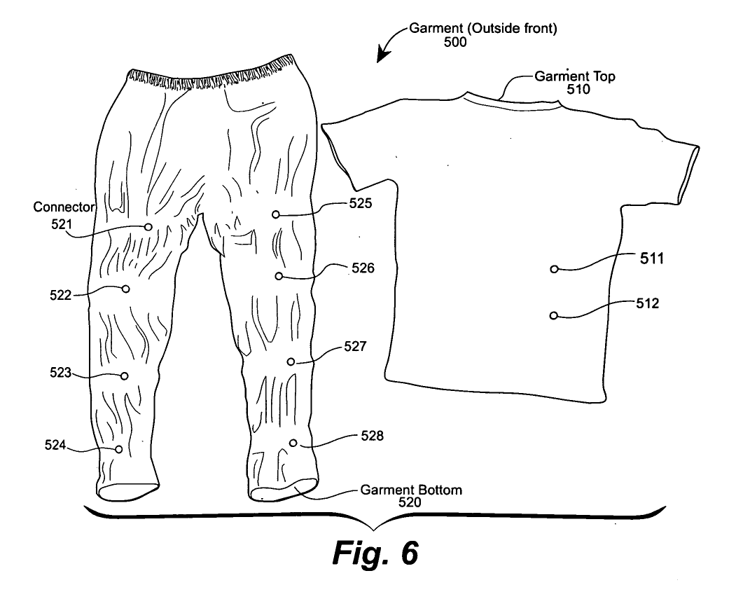

FIG. 6 shows an example of a garment 500 (outside front view). Garment 500 has electrodes (not shown in this view). In this example, garment 500 has two pieces: bottom 520 that covers the wearer's legs, and top 510. Garment 500 has connectors 511, 512, and 521-528 that connect with cables (not shown) and supply electrical current from cables to the electrodes.

FIG. 7 shows an example of a garment 500 (inside front view) having two pieces as mentioned above: top 510, and bottom 520. Garment 500 has electrodes 531-532 and 541-548, and close-fitting fabric adapted to positioning the electrodes in contact with a person's skin. Electrodes 531-532 are adapted to stimulating abdominal muscles. Electrodes 541, 542, 545 and 546 are adapted to stimulating thigh muscles. Electrodes 543, 544, 547, and 548 are adapted to stimulating calf muscles. Garment 500 has connectors 511, 512, and 521-528 that connect with cables and supply electrical current from cables to the electrodes 531-532 and 541-548.

FIG. 8 shows an example of a garment 500 (inside rear view) having two pieces as mentioned above: top 510, and bottom 520. Electrodes 751-754 are adapted to stimulating gluteal muscles. Garment 500 has connectors 731-734 that connect with cables and supply electrical current from cables to the electrodes 751-754. FIG. 8 again shows electrodes 541-548.

In summary, we have shown solutions for responding to G forces and long trip duration, such as methods, systems, kits and garments.

The examples provided herein are intended to demonstrate only some embodiments of the invention. Other embodiments may be utilized and structural changes may be made, without departing from the present invention.

1. A method of responding to G forces, said method comprising:
sensing acceleration; and
electrically stimulating muscle groups of a person subject to said acceleration;
said stimulating being accomplished automatically, with a garment, in an effective amount to raise the blood pressure of said person;
wherein said muscle groups are
abdominal muscles,
gluteal muscles,
thigh muscles, and
calf muscles.

2-24. (canceled)
